(19) United States
(12) Patent Application Publication
(10) Pub. No.: US 2020/0378969 A1
(43) Pub. Date: Dec. 3, 2020

(54) KIT FOR DETECTING SUBSET OF HUMAN REGULATORY T CELL, AND DETECTION METHOD THEREOF

(71) Applicant: SHAOHENG HE, JIN-ZHOU CITY, LIAONING (CN)

(72) Inventors: SHAOHENG HE, JIN-ZHOU CITY, LIANONING PROVINCE (CN); PING HE, SHENYANG, LIAONING (CN)

(21) Appl. No.: 16/348,793

(22) PCT Filed: Jun. 13, 2018
(86) PCT No.: PCT/CN2018/091106

§ 371 Date: May 18, 2020

(30) Foreign Application Priority Data
Apr. 13, 2018 (CN) 201810331146.2

(51) Int. Cl. G01N 33/569; G01N 1/30

(52) U.S. Cl. CPC G01N 33/56972; G01N 1/30; G01N 2333/7051; G01N 2333/70517; G01N 2333/54; G01N 2333/495; G01N 2333/4703; G01N 2333/5428; G01N 2333/715

(57) ABSTRACT

The present invention relates to a kit for detecting a subset of a human regulatory T cell, and a detection method thereof, and belongs to the technical field of cell subset detection. The present invention provides a kit which includes the following components: a blood dilution solution, a mononuclear cell isolation solution, a cell culture solution, a lymphocyte activation liquid, a dead-cell-removing dye, an FcR blocking agent, fluorescence-labeled antibodies against human cell-surface markers, fluorescence-labeled antibodies against human intracellular molecules, a PBS buffer, lipopolysaccharide, a washing buffer, a cell staining buffer, a cell fixing solution, and a membrane-permeable wash buffer. At the time of detection, firstly mononuclear cells are isolated from whole blood, secondly human peripheral blood mononuclear cells are stimulated, and finally the subsets of the regulatory T cells can be detected just by fluorescent antibody staining. Use of the kit and detection method of the present invention can identify 2-6 subsets of regulatory T cells quickly and easily.

### CROSS REFERENCE TO RELATED APPLICATIONS

This application claims priority to the PCT application PCT/CN2018/091106 filed on Apr. 13, 2018 and to Chinese application number 2018103311462, which are incorporated by reference herein in their entirety.

### FIELD OF THE DISCLOSURE

The disclosure relates generally to the field of cell subset detection. More specifically, the disclosure relates to the field of a kit for detecting a subset of a human regulatory T cell, and a detection method thereof.

### BACKGROUND

In mid-1990s, people had proposed a CD4⁺ suppressor T cell, i.e., a regulatory T (Treg) cell, which could inhibit the immunological injury effect of an effector T cell (such as a Th cell) on the human body. At present, more and more data show that there are several different subsets of Treg in human body, and their roles in immune system are different, which makes the action of Treg more complicated.

A naive T cell may differentiate into a variety of regulatory T (Treg) cells under different conditions. Now at least 7 subsets of Treg cells have been identified: (1) nTregs: CD4⁺CD25⁺Foxp3⁺ T cells, which secrete IL-10 and TGF-β, are the most abundant subsets of Treg cells; (2) iTregs: peripheral blood naive CD4⁺ T cells may express Foxp3 as induced by the stimulation of foreign antigens, and Th3 cells which secrete TGF-β and IL-10 also belong to this subset; (3) CD127^{low/-} Treg: CD4⁺ CD25⁺ CD127^{low/-} Foxp3⁺ Treg cells are found in thymus of newborns; (4) ICOS⁺ Treg: ICOS⁺ Treg cells express IL-10, IL-17 and the interferon (IFN)-γ; (5) Tr1 cells: Tr1 cells are CD4⁺ T cells which do not express Foxp3, but secrete IL-10 and may inhibit the function of effector Th cells; (6) CD8⁺ Tregs: OT-1 CD8 cells may quickly differentiate into CD8-expressing and IL-10-secreting Treg cells (CD8⁺ Treg cells) under the action of IL-4 and IL-12, and co-express markers related to activated and naive cells; and (7) IL-17-producing Foxp3⁺ Tregs.

Since there are many subsets of Treg cells and their roles in the body are different, it is particularly important to identify them for individual studies. However currently the commercially available Treg kit only defines the CD4⁺ CD25⁺ Foxp3⁺ T cells as Treg cells, such that CD8⁺ Treg cells and CD127^{low/-} Treg cells are not included, and it may not further identify other Treg cell subsets of among CD4⁺ Treg cells.

### SUMMARY

In some embodiments, an objective is to provide a kit which may simultaneously identify 2-6 a subset of a human regulatory T cell.

A kit for detecting a subset of a human regulatory T cell according to some embodiments includes the following components: a blood dilution solution, a mononuclear cell isolation solution, a cell culture solution, a lymphocyte activation liquid, a dead-cell-removing dye, an FcR blocking agent, fluorescence-labeled antibodies against human cell-surface markers, fluorescence-labeled antibodies against human intracellular molecules, lipopolysaccharide, a washing buffer, a cell staining buffer, a cell fixing solution, and a membrane-permeable washing buffer; where the fluorescence-labeled antibodies against human cell-surface markers includes: an anti-human CD3 or CD4 antibody, an anti-human CD8 antibody, an anti-human CD25 antibody, an anti-human CD127 antibody, an anti-human ICOS antibody, an anti-human TGF-β antibody and an anti-human CCR6 antibody; and the fluorescence-labeled antibodies against human intracellular molecules includes: an anti-human IL-10 antibody, an anti-human IL-17 antibody, and an anti-human Foxp3 antibody.

In some embodiments, the fluorescence species of the dead-cell-removing dye and the fluorescence-labeled antibodies against human cell-surface markers and the fluorescence species of the fluorescence-labeled antibodies against human intracellular molecules independently include: Alexa Fluor 488, Alexa Fluor 594, Alexa Fluor 647, Alexa Fluor 700, APC, APC/Cy7, APC/H7, APC/R700, BB515, BB700, BD IMag/DM, BUV395, BUV496, BUV563, BUV661, BUV737, BUV805, Brilliant Violet 421, Brilliant Violet 480, Brilliant Violet 510, Brilliant Blue 515, Brilliant Violet 570, Brilliant Violet 605, Brilliant Violet 650, Brilliant Violet711, Brilliant Violet750, Brilliant Violet785, Brilliant Violet786, FITC, NA/LE, LEAF, Pacific Blue, PE, PE/CF594, PE/Cy5, PE/Cy7, PE/Dazzle, PerCP, PerCP/Cy 5.5, V450 or V500.

It may be desirable for the fluorescence species to be freely combined according to the laser and optical filter configurations of a flow cytometer.

It may be desirable for the blood dilution solution to include a solution having an osmotic pressure equal to that of human plasma.

It may be desirable for the blood dilution solution to include NaCl solution or PBS buffer.

It may be desirable for the active ingredients of the mononuclear cell isolation solution to include: polysucrose and meglumine diatriazoate, or sodium diatrizoate and polysaccharide, or iodixanol.

It may be desirable for the active ingredients of the cell fixing solution to include formaldehyde and methanol.

It may be desirable for the active ingredients of the membrane-permeable washing buffer to include saponin and fetal bovine serum, or saponin and bovine serum albumin.

It may be desirable for the active ingredients of the FcR blocker to include human immunoglobulin, or an unrelated immunoglobulin which is of the same genus and same subset as those of the antibody used in flow cytometry.

According to an embodiment, a method for detecting a subset of a human regulatory T cell using the above kit includes the steps of:

1) mixing venous blood with the blood dilution solution and then placing the mixture onto the upper layer of the mononuclear cell isolation solution, centrifuging for 15-35 min, and then aspirating mononuclear cells, where the volume ratio of the venous blood, the blood dilution solution and the mononuclear cell isolation solution is 1:(0.5-2):(0.5-2);

2) diluting the mononuclear cells obtained in step 1) with the blood dilution solution, and then centrifuging at 90-600 g for 4-30 min to collect the cell pellet;

3) resuspending the cell pellet obtained in step 2) with the cell culture medium, and adjusting the cell density to 0.5×10⁶-4×10⁶ per mL, so as to obtain the first cell suspension;

4) uniformly mixing the first cell suspension obtained in step 3) with the lymphocyte activation solution and the lipopolysaccharide, and placing the mixture into and cultivating in an incubator containing CO₂ for 4-6 h, to obtain a first incubation cell;

5) mixing the first incubation cell obtained in step 4) with the PBS buffer according to a volume ratio of 1:(0.5-10), then centrifuging at 90-600 g for 4-30 min, and discarding the supernatant to obtain a second cell suspension;

6) taking the second cell suspension obtained in step 5), and mixing it with 0.1-10 μL of DMSO solution containing the dead-cell-removing dye and 0.1-10 μL of the FcR blocker according to 1×10⁵-10×10⁶ cells per 100 μL of volume, and then incubating at 4-40° C. for 5-30 min, so as to obtain a second incubation cell;

7) mixing the second incubation cell obtained in step 6) with 0.5-5 mL of the cell staining buffer according to 1×10⁵-10×10⁶ cells per 100 μL of volume, centrifuging at 90-600 g for 4-30 min, discarding the supernatant, and then adding 0.5-20 μL of each of the fluorescence-labeled antibodies against human cell-surface markers into the cell pellet according to 1×10⁵-10×10⁶ cells per 100 μL of volume, uniformly mixing, and then incubating with protection from light at 4-40° C. for 5-30 min;

8) mixing the third incubation cell obtained in step 7) with 0.5-5 mL of the cell staining buffer according to 1×10⁵-10×10⁶ cells per 100 μL of volume, then centrifuging at 90-600 g for 4-30 min, discarding the supernatant, then adding 0.1-5 mL of the cell fixing solution into the pellet according to 1×10⁵-10×10⁶ cells per 100 μL of volume for resuspension, and incubating with protection from light at 4-40° C. for 10-120 min, so as to obtain resuspension of the third incubation cell;

9) mixing the cell suspension of the third incubation cell obtained in step 8) with 0.5-5 mL of the membrane-permeable washing buffer according to 1×10⁵-10×10⁶ cells per 100 μL of volume, centrifuging at 90-600 g for 4-30 min, discarding the supernatant to obtain a pellet of the third incubation cell, and then again adding 0.5-5 mL of the membrane-permeable washing buffer according to 1×10⁵-10×10⁶ cells per 100 μL of volume to resuspend the pellet of the third incubation cell, centrifuging at 90-600 g for 4-30 min, and discarding the supernatant to obtain a pellet of the third incubation cell;

10) adding 0.5-20 μL of each of the fluorescence-labeled antibodies against human intracellular molecules into the pellet of the third incubation cells obtained in step 9) according to 1×10⁵-10×10⁶ cells per 100 μL of volume, uniformly mixing, and then incubating with protection from light at 4-40° C. for 10-120 min, so as to obtain a fourth incubation cells; and

11) adding 0.5-5 mL of the membrane-permeable washingbuffer into the pellet of the fourth incubation cell obtained in step 10) according to 10⁵-10⁶ cells per 100 μL of volume, uniformly mixing, centrifuging at 90-600 g for 4-30 min, and discarding the supernatant to obtain a pellet of the fourth incubation cells, adjusting the cell density to (0.1-10)×10⁶ per mL with PBS, and then detecting the antibody expression by a flow cytometer; and obtaining the detection result of the Treg cell subsets according to the antibody expression, where the correspondence relationship between the Treg cell subset and the antibody expression may be as follows:

| Serial | | Expression conditions of |
|---|---|---|
| Number | Subset of Treg Cell | antibodies |
| 1 | Inducible (or adaptive) Treg | CD4⁺ CD25⁺ Foxp3⁺ |
| | (iTreg) | IL-10⁺ TGF-β⁺ |
| 2 | CD127 low-expression and negative | CD4⁺ CD25⁺Foxp3⁺ |
| | Treg (CD127^{(low&-)} Treg) | CD127^{(low&-)} |
| 3 | ICOS positive Treg (ICOS⁺ | CD4⁺ CD25⁺Foxp3⁺ |
| | Treg) | ICOS⁺ |
| 4 | IL-10-producing Treg Type 1 | CD4⁺ CD25⁺Foxp3⁻ |
| | (Tr1) | IL-10⁺ |
| 5 | CD8 positive Treg (CD8⁺ | CD8⁺ CD25⁺ Foxp3⁺ |
| | Treg) | |
| 6 | IL-17-producing Treg (IL-17⁺ | CD4⁺ Foxp3⁺ CCR6⁺ |
| | Treg) | IL-17⁺ |

Note:
+ represents positive; low represents low expression; and-represents negative.

In some embodiments, the centrifugal conditions of step 1) includes: a centrifugal force of 200-1000 g, and a centrifugal temperature of 18-22° C. In step 2) the volume ratio of the mononuclear cell to the blood dilution solution is 1:(0.5-50). The cell culture solution of step 3) is based on an RPMI 1640 medium and includes fetal bovine serum at a volume concentration of 0.1%-20% and a penicillin-streptomycin double-resistant solution at a volume concentration of 0-3%. The incubation conditions of step 4) include incubating in an incubator containing 5% CO₂ at an incubation temperature of 35-40° C. The addition amount of each one of the fluorescence-labeled antibodies against human cell-surface markers and fluorescence-labeled antibodies against human intracellular molecules of step 7) and step 9) is determined according to the number of incubation cells and the working concentration of the antibody.

In further embodiments, the present invention provides a kit for detecting a subset of a human regulatory T cell and a detection method thereof. The kit includes simple and readily available components, and may simultaneously identify 2-6 subsets of Treg cells in human blood.

### BRIEF DESCRIPTION OF THE DRAWINGS

Illustrative embodiments of the present disclosure are described in detail below with reference to the attached drawing figures.

FIG. 1 shows a human peripheral blood mononuclear cell population.

FIG. 2 shows a human peripheral blood mononuclear cell population after removal of dead cells.

FIG. 3 shows the expression of CD4+ (CD3+CD8−) cells and CD8+ (CD3+ CD8−) cells in the human peripheral blood mononuclear cell population.

FIG. 4 shows the expression of CD8+Treg: CD8+CD25+ Foxp3+ cells in human peripheral blood CD3+ CD8+ cell population.

FIG. 5 shows the expression of CD25 and Foxp3 in human peripheral blood CD3+ CD8− cell population.

FIG. 6 shows the expression of inducible (or adaptive) Treg (iTreg): CD4+CD25+ Foxp3+ IL-10+ TGF-β+ cells in human peripheral blood CD3+ CD8− CD25+ Foxp3+ cell population.

FIG. 7 shows the expression of IL-10-producing type 1 Treg (Tr1):CD4+ CD25+ Foxp3− IL-10+ cells in the human peripheral blood CD3+ CD8− CD25+ Foxp3− cell population.

FIG. 8 shows the expression of CD127 low-expression and negative Treg (CD127(low&-) Treg): CD4+ CD25+ Foxp3+ CD127(low&-) cells in human peripheral blood CD3+ CD8− CD25+ Foxp3+ cell population.

FIG. 9 shows the expression of ICOS positive Treg (ICOS+ Treg): CD4+ CD25+ Foxp3+ ICOS+ cells in human peripheral blood CD3+ CD8− CD25+ Foxp3+ cell population.

FIG. 10 shows the expression of IL-17-producing Treg (IL-17+ Treg):CD4+ Foxp3+ CCR6+ IL-17+ cells in human peripheral blood CD3+ CD8− Foxp3+ cell population.

### DETAILED DESCRIPTION

Some embodiments of the present invention are further described below with reference to the accompanying drawings and embodiments.

In some embodiments, the present invention provides a kit for detecting a subset of a human regulatory T cell, which includes the following components: a blood dilution, a mononuclear cell isolation solution, a cell culture solution, a lymphocyte activation liquid, a dead-cell-removing dye, an FcR blocking agent, fluorescence-labeled antibodies against human cell-surface markers, fluorescence-labeled antibodies against human intracellular molecules, a PBS buffer, lipopolysaccharide, a washing buffer, a cell staining buffer, a cell fixing solution, and a membrane-permeable wash buffer.

In embodiments, the fluorescence-labeled antibodies against human cell-surface markers include: an anti-human CD3 or CD4 antibody, an anti-human CD8 antibody, an anti-human CD25 antibody, an anti-human CD127 antibody, an anti-human TGF-β antibody, an anti-human ICOS antibody and an anti-human CCR6 antibody; and the fluorescence-labeled antibodies against human intracellular molecules include: an anti-human IL-10 antibody, an anti-human IL-17 antibody, and an anti-human Foxp3 antibody.

In embodiments, the fluorescence species of the dead-cell-removing dye and the fluorescence-labeled antibodies against human extracellular molecules, and the fluorescence species of the fluorescence-labeled antibodies against human intracellular molecules independently and may include: Alexa Fluor 488, Alexa Fluor 594, Alexa Fluor 647, Alexa Fluor 700, APC, APC/Cy7, APC/H7, APC/R700, BB515, BB700, BD IMag/DM, BUV395, BUV496, BUV563, BUV661, BUV737, BUV805, Brilliant Violet 421, Brilliant Violet 480, Brilliant Violet 510, Brilliant Blue 515, Brilliant Violet 570, Brilliant Violet 605, Brilliant Violet 650, Brilliant Violet711, Brilliant Violet750, Brilliant Violet785, Brilliant Violet786, FITC, NA/LE, LEAF, Pacific Blue, PE, PE/CF594, PE/Cy5, PE/Cy7, PE/Dazzle, PerCP, PerCP/Cy 5.5, V450 or V500.

In embodiments, the fluorescence species may be freely combined according to the laser and optical filter configurations of a flow cytometer.

In embodiments, the blood dilution may be a solution having an osmotic pressure equal to that of human plasma, and particularly the blood dilution solution may include a NaCl solution or a PBS buffer, where the mass concentration of the NaCl solution is 0.9%; according to an embodiment, the PBS buffer includes the following components in parts by weight: 8.0 parts of NaCl, 0.20 parts of KCl, 1.16 parts of Na2HPO4 and 0.20 parts of KH2PO4; and the pH value of the PBS buffer is 7.0-7.6, and preferably 7.2-7.5. The present invention has no specific limitation on the source of the blood dilution solution.

In embodiments, the active ingredients of the mononuclear cell isolation solution may include: polysucrose and meglumine diatriazoate, or sodium diatrizoate and polysaccharide, or iodixanol, and the solvent of the mononuclear cell isolation solution is distilled water. The present invention has no specific limitation on the source and specie of the mononuclear cell isolation solution, and the mononuclear cell isolation solution has a density of 1.076-1.078 g/mL, and an osmotic pressure of 275-305 mOsm.

In embodiments, the invention has no specific limitation on the specie and source of the cell culture solution. The cell culture solution may be a culture solution for culturing human peripheral blood mononuclear cells, and the culture solution may be based on an RPMI 1640 medium and contains fetal bovine serum and a penicillin-streptomycin double-resistant solution. According to an embodiment, the final volume concentration of the fetal bovine serum may be 0.1-20%, preferably 0.5-15%, and more preferably 1-10%. According to an embodiment, the final volume concentration of the penicillin-streptomycin double-resistant solution may be 0-3%, and preferably 0.5-2%.

In embodiments, the active ingredients of the lymphocyte activation solution may include: phorbol myristate acetate, ionomycin and monensin, or alternatively phorbol myristate acetate, ionomycin and brefeldin A, or alternatively phorbol myristate acetate, calcium ionophore and monensin, or alternatively phorbol myristate acetate, calcium ionophore and brefeldin A; according to an embodiment, the concentration of the lymphocyte activation solution may be 500-5,000 times greater than that of a working solution during use; the working concentration of the phorbol myristate acetate may be 0.01-0.50 μM, preferably 0.050-0.2 μM; the working concentration of the ionomycin may be 0.5-5.0 μM, and preferably 1.0-4.0 μM; the working concentration of the monensin may be 0.5-6.0 μM, and preferably 1.0-3.0 μM; the working concentration of the brefeldin A may be 0.5-30 μg/mL, and preferably 1-20 μg/mL; and the working concentration of calcium ionophore may be 25-1000 ng/mL, and preferably 100-500 ng/mL. According to an embodiment, the solvent of the lymphocyte activation solution may be DMSO.

In embodiments, the dead-cell-removing dye may include an amine reactive fluorescent dye. The amine reactive fluorescent dye may penetrate the cell membrane of a damaged cell, and may not penetrate the cell membrane of an intact cell. According to an embodiment, the dead-cell-removing dye may be a solution obtained by dissolving the amine reactive fluorescent dye in DMSO.

In embodiments, the fluorescence-labeled antibodies against human cell-surface markers include: an anti-human CD3 or CD4 antibody, an anti-human CD8 antibody, an anti-human CD25 antibody, an anti-human CD127 antibody, an anti-human ICOS antibody, an anti-human TGF-β antibody and an anti-human CCR6 antibody; and the fluorescence-labeled antibodies against human intracellular molecules include: an anti-human IL-10 antibody, an anti-human IL-17 antibody, and an anti-human Foxp3 antibody.

In embodiments, the fluorescence species of the dead-cell-removing dye, the fluorescence-labeled antibodies against human extracellular molecules, and the fluorescence-labeled antibodies against human intracellular molecules respectively include: Alexa Fluor 488, Alexa Fluor 594, Alexa Fluor 647, Alexa Fluor 700, APC, APC/Cy7, APC/H7, APC/R700, BB515, BB700, BD IMag/DM, BUV395, BUV496, BUV563, BUV661, BUV737, BUV805, Brilliant Violet 421, Brilliant Violet 480, Brilliant Violet 510, Brilliant Blue 515, Brilliant Violet 570, Brilliant Violet 605, Brilliant Violet 650, Brilliant Violet711, Brilliant Violet750, Brilliant Violet785, Brilliant Violet786, FITC, NA/LE, LEAF, Pacific Blue, PE, PE/CF594, PE/Cy5, PE/Cy7, PE/Dazzle, PerCP, PerCP/Cy 5.5, V450 or V500, etc.

In embodiments, the fluorescence species may be freely combined according to the laser and optical filter configurations of a flow cytometer.

In embodiments, the washing buffer may include a PBS buffer.

In embodiments, the cell staining buffer may be a calcium-free and magnesium-free Dulbecco's PBS (DPBS) solution, and the active ingredient of the DPBS solution may be fetal bovine serum, where the working concentration of the fetal bovine serum of according to an embodiment may be 0.1-20%, preferably 0.5-15%, and more preferably 1-10%. According to an embodiment, the pH value of cell staining buffer may be 7.0-7.6.

In embodiments, the active ingredient of the cell fixing solution may include formaldehyde and methanol, the working concentration of formaldehyde may be 0.5%-10% (mass/mass), and preferably 0.8%-2% (mass/mass); and the working concentration of methanol may be 0.1%-2% (mass/mass), and preferably 0.2%-1% (mass/mass).

In embodiments, the active ingredient of the membrane-permeable washing buffer may include saponin and fetal bovine serum, or saponin and bovine serum albumin, where the concentration of the saponin may be 0.1-5% (w/v); the volume concentration of the fetal bovine serum may be 0.1-20%; and the mass volume concentration of the bovine serum albumin may be 0.1-20%.

In embodiments, the active ingredients of the FcR blocker may include a human immunoglobulin, or an unrelated immunoglobulin which may be of the same genus and same subset as those of the antibody used in the flow cytometry. The mass concentration of the working solution of the FcR blocker may be 0.1-100 μg/mL, and preferably 1-10 μg/mL.

In further embodiments, the present invention may provide a method for detecting a subset of a human regulatory T cell using the aforementioned kit, including the steps of:

1) mixing venous blood with the blood dilution solution and then placing the mixture onto the upper layer of the mononuclear cell isolation solution, centrifuging for 15-35 min, and then aspirating mononuclear cells, where the volume ratio of the venous blood, the blood dilution solution and the mononuclear cell isolation solution may be 1:(0.5-2):(0.5-2);

2) diluting the mononuclear cells obtained in step 1) with the blood dilution solution, and then centrifuging at 90-600 g for 4-30 min to collect the cell pellet;

3) resuspending the cell pellet obtained in step 2) with the cell culture medium, and adjusting the cell density to 0.5×10⁶-4×10⁶ per mL, so as to obtain the first cell suspension;

4) uniformly mixing the first cell suspension obtained in step 3) with the lymphocyte activation solution and the lipopolysaccharide, and placing the mixture into and cultivating in an incubator containing CO₂ for 4-6 h, to obtain a first incubation cell;

5) mixing the first incubation cell obtained in step 4) with the PBS buffer according to a volume ratio of 1:(0.5-10), then centrifuging at 90-600 g for 4-30 min, and discarding the supernatant to obtain a second cell suspension;

6) taking the second cell suspension obtained in step 5) and mixing it with 0.1-10 μL of a DMSO solution containing the dead-cell-removing dye and 0.1-10 μL of the FcR blocker according to 1×10⁵-10×10⁶ cells per 100 μL of volume, and then incubating at 4-40° C. for 5-30 min, so as to obtain a second incubation cell;

7) mixing the second incubation cell obtained in step 6) with 0.5-5 mL of the cell staining buffer according to 1×10⁵-10×10⁶ cells per 100 μL of volume, centrifuging at 90-600 g for 4-30 min, discarding the supernatant, and then adding 0.5-20 μL of each of the fluorescence-labeled antibodies against human cell-surface markers into the cell pellet according to 1×10⁵-10×10⁶ cells per 100 μL of volume, uniformly mixing, and then incubating with protection from light at 4-40° C. for 5-30 min;

8) mixing the third incubation cell obtained in step 7) with 0.5-5 mL of the cell staining buffer according to 1×10⁵-10×10⁶ cells per 100 μL of volume, then centrifuging at 90-600 g for 4-30 min, discarding the supernatant, then adding 0.1-5 mL of the cell fixing solution into the pellet according to 1×10⁵-10×10⁶ cells per 100 μL of volume for resuspension, and incubating with protection from light at 4-40° C. for 10-120 min, so as to obtain resuspension of the third incubation cell;

9) mixing the resuspension of the third incubation cell obtained in step 8) with 0.5-5 mL of the membrane-permeable washing buffer according to 1×10⁵-10×10⁶ cells per 100 μL of volume, centrifuging at 90-600 g for 4-30 min, discarding the supernatant to obtain a pellet of the third incubation cell, and then again adding 0.5-5 mL of the membrane-permeable washing buffer according to 1×10⁵-10×10⁶ cells per 100 μL of volume to resuspend the pellet of the third incubation cell, centrifuging at 90-600 g for 4-30 min, and discarding the supernatant to obtain a pellet of the third incubation cell;

10) adding 0.5-20 μL of each of the fluorescence-labeled antibodies against human intracellular molecules into the pellet of the third incubation cell obtained in step 9) according to 1×10⁵-10×10⁶ cells per 100 μL of volume, uniformly mixing, and then incubating with protection from light at 4-40° C. for 10-120 min, so as to obtain a fourth incubation cell; and

11) adding 0.5-5 mL of the membrane-permeable wash buffer into the pellet of the fourth incubation cell obtained in step 10) according to 1×10⁵-10×10⁶ cells per 100 μL of volume, uniformly mixing, centrifuging at 90-600 g for 4-30 min, and discarding the supernatant to obtain a pellet of the fourth incubation cell, adjusting the cell density to (0.1-10)×106 per mL with PBS, and then detecting the antibody expression by flowcytometer; and obtaining the detection result of the Treg cell subset according to the antibody expression, where the correspondence relationship between the Treg cell subset and the antibody expression may be as follows:

| Serial | | Expression conditions of |
|---|---|---|
| Number | Subset of Treg Cell | antibodies |
| 1 | Inductive (or adaptive) Treg | CD4⁺ CD25⁺ Foxp3⁺ |
| | (iTreg) | IL-10⁺ TGF-β⁺ |
| 2 | CD127 low-expression and negative | CD4⁺CD25⁺ Foxp3⁺ |
| | Treg (CD127^{(low&-)} Treg) | CD127^{(low&-)} |
| 3 | ICOS positive Treg (ICOS⁺ | CD4⁺ CD25⁺ Foxp3⁺ |
| | Treg) | ICOS⁺ |
| 4 | IL-10-producing Treg type 1 | CD4⁺ CD25⁺ Foxp3⁻ |
| | (Tr1) | IL-10⁺ |
| 5 | CD8 positive Treg (CD8⁺ | CD8⁺ CD25⁺ Foxp3⁺ |
| | Treg) | |
| 6 | IL-17-producing Treg (IL-17⁺ | CD4⁺ Foxp3⁺ CCR6⁺ |
| | Treg) | IL-17⁺ |

Note:
+ represents positive; low represents low expression; and-represents negative.

In embodiments, the venous blood may be mixed with the blood dilution solution and then placed onto the upper layer of the mononuclear cell isolation solution, centrifuged for 15-35 min, and then mononuclear cells are aspirated. According to an embodiment, the volume ratio of the venous blood, the blood dilution solution and the mononuclear cell isolation solution may be 1:(0.5-2.0):(0.5-2.0), preferably 1:(0.9-1.1):(0.9-1.1), and more preferably 1:1:1. According to an embodiment, the mixing may be mixing the venous blood and the blood dilution solution according to the volume ratio of 1:1, and then placing the mixture onto the upper layer of the mononuclear cell isolation solution. According to an embodiment, during the centrifuging the acceleration may be set to 9 and the deceleration may be set to 0; the centrifugal force may be 200-1000 g, preferably 250-950 g, and more preferably 300-900 g; the centrifugal time may be 15-35 min, where the centrifugal time may be chosen to 20-40 min when the venous blood may be stored for >2 h; and the temperature for the centrifuging may be 18-22° C. The liquid may be stratified after centrifuging, and then mononuclear cells are obtained by carefully aspirating the white film layer on the interface with a pipette, trying not to aspirate the liquid in the upper layer.

In embodiments, after the mononuclear cells are obtained, the mononuclear cells may be diluted with the blood dilution solution and then centrifuged for 4-30 min to collect the cell pellet. According to an embodiment, the volume ratio of the mononuclear cells to the blood dilution solution may be 1:(0.5-50). According to an embodiment, the centrifugal time may be 4-30 min, and the centrifugal force for centrifuging may be 90-600 g, preferably 100-550 g, and more preferably 150-500 g.

In embodiments, after the cell pellet may be collected, the obtained cell pellet may be suspended with the cell culture medium, and the cell density may be adjusted to 0.5×10⁶-4×10⁶ per mL, so as to obtain the first cell suspension. According to an embodiment, the cell culture medium may be added in twice, where the addition for the first time may be used for resuspending the cell pellet, and the volume of the medium added for the first time may be 0.1-1.0 mL/(peripheral blood mononuclear cells prepared from 1 mL whole bloo4), and then the cells are counted; and then the medium may be added for the second time to achieve a final cell density of 0.5×10⁶-4×10⁶ per mL.

In embodiments, after the first cell suspension may be obtained, the first cell suspension may be mixed with 0.1-10 μL of the lymphocyte activation solution and 0.1-10 μL of the lipopolysaccharide according to 1×10⁵-10×10⁶ cells per 100 μL of volume, and then incubated for 4-6 h to obtain the first incubation cell. According to an embodiment, the mixing may be pipetting up and down with a pipette, and the number of pipetting up and down conducted with the pipette may be 5-20, preferably 5-15, and more preferably 8-12. According to an embodiment, the incubation may be conducted in a CO₂ incubator, and the volume concentration of CO₂ in the CO₂ incubator may be 3%-8%, and preferably 4.5%-5.5%. according to an embodiment, the temperature in the CO₂ incubator may be 35-40° C., preferably 36-38° C., and more preferably 36.5-37.5° C.

In embodiments, after the first incubation cell may be obtained, the culture cell may be mixed with the PBS buffer in a volume ratio of 1:(0.5-50), and centrifuged for 4-30 min, and the supernatant may be discarded to obtain a second cell suspension. According to an embodiment, the centrifugal rotation speed may be 90-600 g, preferably 100-550 g, and more preferably 150-500 g; and the centrifugal time may be 4-30 min, and preferably 5-20 min. According to an embodiment, the density of the first incubation cell may be adjusted after the centrifuging, and the adjustment may be conducted by using the PBS buffer.

In embodiments, after the second cell suspension may be obtained, the second cell suspension may be mixed with 0.1-10 μL of a DMSO solution containing the dead-cell-removing dye and 0.1-10 μL of the FcR blocker according to 1×10⁵-10×10⁶ cells per 100 μL of volume, and then incubated for 5-30 min, so as to obtain a second incubation cell. According to an embodiment, the dissolved dead-cell-removing dye may be dissolved in DMSO. According to an embodiment, the incubation temperature may be 4-40° C., and preferably 15-35° C.; the incubation may be conducted in an environment with the protection from light; and the incubation time may be 5-30 min, and preferably 10-20 min.

In embodiments, after the second incubation cell may be obtained, the second incubation cell may be mixed with 0.5-5 mL of the cell staining buffer according to 1×10⁵-10×10⁶ cells per 100 μL of volume and centrifuged for 4-30 min, and the supernatant may be discarded, and then 0.5-20 μL of each of the fluorescence-labeled antibodies against human cell surface markers may be added into the cell pellet according to 1×10⁵-10×10⁶ cells per 100 μL of volume, uniformly mixed, and then incubated, so as to obtain the third incubation cell. According to an embodiment, the centrifugal rotation speed may be 90-600 g, preferably 100-550 g, and more preferably 150-500 g, and the centrifugal time may be 4-30 min, and preferably 5-20 min. According to an embodiment, the incubation temperature may be 4-40° C., preferably 15-35° C., and more preferably 18-28° C.; the incubation may be conducted in an environment with the protection from light; and the incubation time may be 5-30 min, and preferably 10-20 min.

In embodiments, after the third incubation cell may be obtained, the third incubation cell may be mixed with 0.5-5 mL of the cell staining buffer according to 1×10⁵-10×10⁶ cells per 100 μL of volume and centrifuged at 90-600 g for 4-30 min, the supernatant may be discarded, and then the cell fixing solution may be added into the cell pellet for resuspension, and incubated for 10-120 min, so as to obtain resuspension of the third incubation cell. According to an embodiment, the volume ratio of the third incubation cell to the cell fixing solution may be according to 1×10⁵-10×10⁶ cells per 100 μL of volume: 0.1-5.0 mL. According to an embodiment, the centrifugal rotation speed may be 90-600 g, preferably 100-550 g, and more preferably 150-500 g, and the centrifugal time may be 4-30 min, and preferably 10-20 min. According to an embodiment, the incubation temperature may be 4-40° C., preferably 15-35° C., and more preferably 18-28° C.; the incubation may be conducted in an environment with the protection from light; and the incubation time may be 10-150 min, and preferably 20-120 min.

In embodiments, after the resuspension of the third incubation cell may be obtained, the resuspension of the third incubation cell may be mixed with the membrane-permeable wash buffer according to the volume ratio of 1×10⁵-10×10⁶ cells per 100 μL of volume:(0.1-5 mL) and centrifuged for 4-30 min, and the supernatant may be discarded, so as to obtain the pellet of the third incubation cell. According to an embodiment, the volume ratio of the suspension of the third incubation cell to the membrane-permeable wash buffer may be 1×10⁵-10×10⁶ cells per 100 μL of volume: (0.1-5 mL), and preferably 1×10⁵-10×10⁶ cells per 100 μL of volume:(0.2-4 mL). According to an embodiment, the centrifugal rotation speed may be 90-600 g, preferably 100-550 g, and more preferably 150-500 g, and the centrifugal time may be 4-30 min, and preferably 5-20 min.

In embodiments, after the pellet of the third incubation cell may be obtained, the pellet of the third incubation cell may be mixed with (0.1-20) μL of each of the fluorescence-labeled antibodies against human intracellular molecules according to 1×10⁵-10×10⁶ cells per 100 μL of volume and centrifuged for 10-120 min, so as to obtain a fourth incubation cell.

In embodiments, the pellet of the third incubation cell may be washed before the pellet of the third incubation cell may be mixed with the membrane-permeable washing buffer and the fluorescence-labeled antibodies against human intracellular molecules. According to an embodiment, the number of washing may be 1-3, and preferably 2; the washing may be conducted with the membrane-permeable wash buffer at each time of washing; according to 1×10⁵-10×10⁶ cells per 100 μL of cell suspension, the dosage of the membrane-permeable wash buffer may be 0.5-5 mL, preferably 0.5-4 mL, and more preferably 1-3 mL; and centrifuging may be conducted after the washing, and the supernatant may be discarded to obtain the washed pellet of the third incubation cell. According to an embodiment, the centrifugal rotation speed may be 90-600 g, preferably 100-550 g, and more preferably 150-500 g, and the centrifugal time may be 4-30 min, and more 5-20 min.

In embodiments, after the washed pellet of the third incubation cell may be obtained, the washed pellet of the third incubation cell may be mixed with the fluorescence-labeled antibodies against human intracellular molecules according to the proportion of 1×10⁵-10×10⁶ cells per 100 μL of cell suspension: (0.1-20 μL), and then incubated for 10-120 min, so as to obtain the fourth incubation cell. According to an embodiment, the volume ratio of the washed pellet of the third incubation cell to the fluorescence-labeled antibodies against human intracellular molecules may be 1×10⁵-10×10⁶ cells per 100 μL of cell suspension: (0.1-20 μL), and preferably 1×10⁵-10×10⁶ cells per 100 μL of cell suspension: (0.5-15 μL). According to an embodiment, the incubation may be conducted under the conditions with protection from light, the incubation temperature may be 4-40° C., preferably 15-35° C., and more preferably 18-28° C.; and the incubation time may be 10-120 min, and more 20-100 min.

In embodiments, after the fourth incubation cell may be obtained, the fourth incubation cell may be mixed with the membrane-permeable washing buffer according to 1×10⁵-10×10⁶ cells per 100 μL of cell suspension: (0.5-5 mL) and then centrifuged, and the antibody expression may be detected by a flow cytometer. According to an embodiment, the volume ratio of the fourth incubation cell, the membrane-permeable washing buffer and the cell staining buffer may be 1×10⁵-10×10⁶ cells per 100 μL of cell suspension: (0.5-5 mL), and preferably 1×10⁵-10×10⁶ cells per 100 μL of cell suspension: (0.5-4 mL). According to an embodiment, the antibody expression may be detected by a flow cytometer, the Treg cell subsets are identified according to the antibody expression, and the details may be as follows:

| Serial | | Expression conditions of |
|---|---|---|
| Number | Subset of Treg Cell | antibodies |
| 1 | Inductive (or adaptive) Treg | CD4⁺ CD25⁺Foxp3⁺ |
| | (iTreg) | IL-10⁺ TGF-β⁺ |
| 2 | CD127 low-expression and negative | CD4⁻ CD25⁺Foxp3⁺ |
| | Treg (CD127^{(low&-)} Treg) | CD127^{(low&-)} |
| 3 | ICOS positive Treg (ICOS⁺ | CD4⁺ CD25⁺Foxp3⁺ |
| | Treg) | ICOS⁺ |
| 4 | IL-10-producing Treg type 1 | CD4⁺CD25⁺ Foxp3⁻ |
| | (Tr1) | IL-10⁺ |
| 5 | CD8 positive Treg (CD8⁺ | CD8⁺ CD25⁺ Foxp3⁺ |
| | Treg) | |
| 6 | IL-17-producing Treg (IL-17⁺ | CD4⁺ Foxp3⁺ CCR6⁺ |
| | Treg) | IL-17⁺ |

Note:
+ represents positive; low represents low expression; and-represents negative.

In some embodiments, the kit for detecting a subset of a human regulatory T cell as provided may be described in detail in connection with the following embodiments, but they should not be construed as limiting the claimed scope of the present invention.

### Embodiment 1

Separation of mononuclear cells from whole blood

1) 1 mL of fresh venous blood (stored for ≤2 h) was collected and diluted with 1 mL of the blood dilution solution;

2) 1 mL of the mononuclear cell isolation solution was taken and put at the bottom of a centrifuge tube, the diluted blood was carefully laid on top of the mononuclear cell isolation solution without mixing the blood and the mononuclear cell isolation solution;

3) they were centrifuged in a centrifugal machine at 800 g for 20 min, where for the centrifugal machine the temperature was set to 18° C., the acceleration was set to 9, and the deceleration was set to 0;

4) the white film layer (mononuclear cells) on the interface was aspirated with a pipette, trying not to aspirate the liquid in the upper layer; and

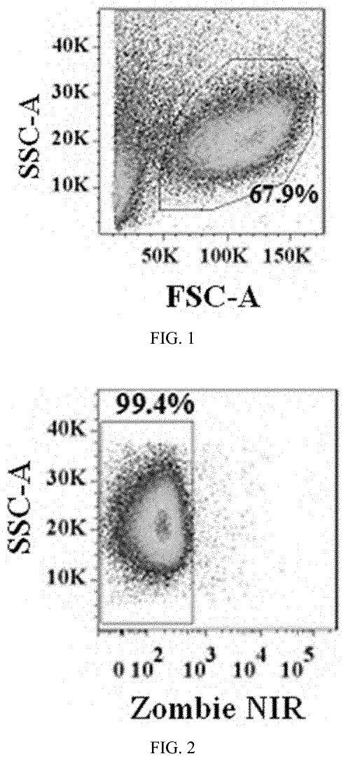

5) the solution of mononuclear cells was diluted with 2 mL of the blood dilution solution, and centrifuged at 250 g under room temperature for 10 min; the cell pellet was collected, and the mononuclear cells were resuspended in 100 μL of the cell culture medium, so as to obtain the mononuclear cells as shown in FIG. 1.

### Embodiment 2

Stimulation to human peripheral blood mononuclear cells:

1) cells were counted, and the cell density was adjusted to 1×10⁶ per mL with the cell culture medium;

2) 2 μL of the lymphocyte activation solution and 1 μL of LPS were added per 1 mL of the cell suspension, uniformly mixed and then plated onto a cell culture plate, and placed into a CO₂ incubator to incubate at 37° C. for 4 h;

3) 1 mL of cells were collected per tube, added with 1 mL of a phosphate buffer, and centrifuged at 200 g for 6 min; and

4) cells were counted, and the cell density was adjusted to 1×10⁷ per mL of the cell suspension with the phosphate buffer.

### Embodiment 3

Fluorescent Antibody Staining:

1) 100 μL of the cell suspension was added into each tube, then added with 1 μL of the dissolved dead-cell-removing dye and 5 μL of the FcR blocker, and incubated with protection from light at room temperature for 15 min;

2) 1 mL of the cell staining buffer was added into each tube and centrifuged at 200 g for 6 min, and the supernatant was discarded; and the obtained human peripheral blood mononuclear cell population after the removal of dead cells was as shown in FIG. 2;

3) 100 μL of the cell staining buffer was added into each tube for cell resuspension, then 5 μL of each of a PerCP/Cy 5.5-labeled anti-human CD3 antibody, a FITC-labeled anti-human CD8 antibody, a PE-labeled anti-human CD25 antibody, a Brilliant Violet 421-labeled anti-human TGF-β antibody and a Brilliant Violet 510-labeled anti-human CD127 antibody, or 5 μL of each of a PerCP/Cy 5.5-labeled anti-human CD3 antibody, a FITC-labeled anti-human CD8 antibody, a Brilliant Violet 510-labeled anti-human CD25 antibody, a PE/Cy7-labeled anti-human CD278 (ICOS) antibody and a PE-labeled anti-human CCR6 antibody was added, and incubated with protection from light at room temperature for 15 min;

4) 1 mL of the cell staining buffer was added into each tube and centrifuged at 200 g for 6 min, and the supernatant was discarded;

5) 1 mL of the fixing membrane-permeable washing buffer was added into each tube for cell resuspension, and incubated with protection from light for 30 min;

6) 2 mL of the membrane-permeable washing buffer was added into each tube and centrifuged at 200 g for 6 min, and the supernatant was discarded;

7) 100 μL of the membrane-permeable washing buffer was added into each tube for cell resuspension, then 5 μL of each of an Alexa Fluor 647-labeled anti-human Foxp3 antibody, a PE/Cy 7-labeled anti-human IL-10 antibody, or 5 μL of each of a Brilliant Violet 421-labeled anti-human IL-17A antibody and an Alexa Fluor 647-labeled anti-human Foxp3 antibody was added, and incubated with protection from light at room temperature for 1 h; and

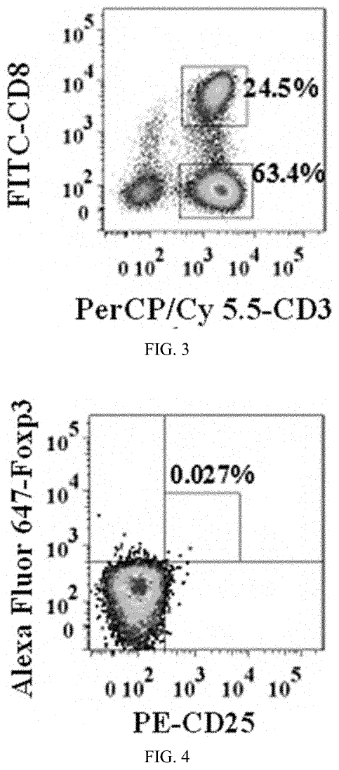

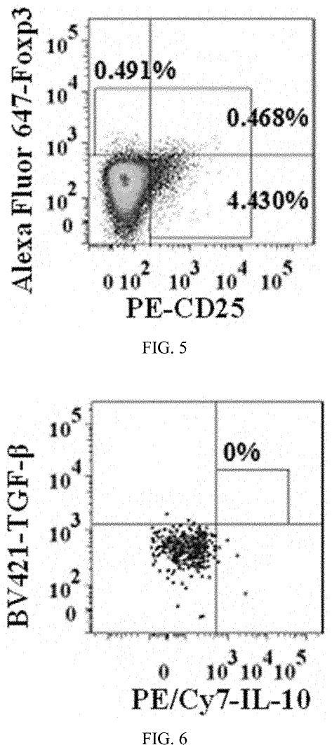

8) 2 mL of the membrane-permeable wash buffer was added into each tube to wash for once, 200 μL of the cell staining buffer was used to resuspend the cells, the cell resuspension was loaded onto a flow cytometer to detect the expression conditions of respective molecules, and the expression conditions of respective antibodies were as shown in FIGS. 3-10.

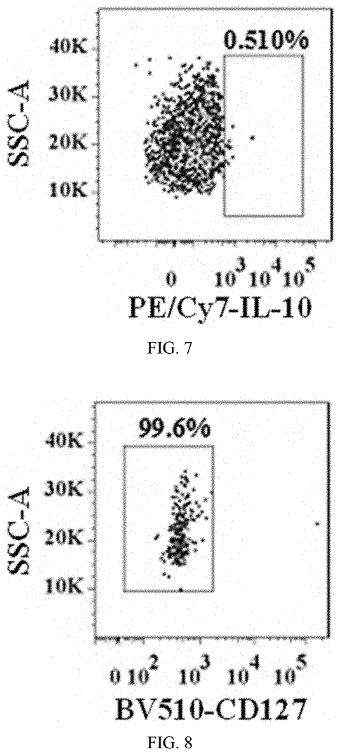

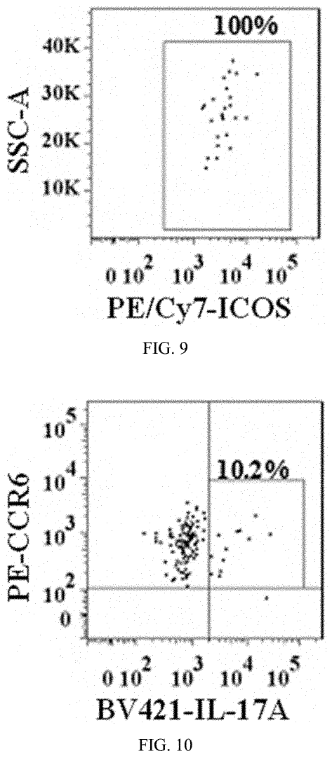

It may be seen from the above Embodiments that, the kit and the detection method thereof according to an embodiment may rapidly and simultaneously detect 2-6 subsets of Treg cells in human blood, where the expression of the inducible (or adaptive) Treg is shown in FIG. 6; the expression of the IL-10-producing Treg is shown in FIG. 7; the expression of the CD127-low-expression and negative Treg is shown in FIG. 8; the expression of the ICOS positive Treg is shown in FIG. 9; and the expression of the IL-17-producing Treg is shown in FIG. 10.

The foregoing descriptions are only some implementation manners of the present invention. It should be noted that for a person of ordinary skill in the art, several improvements and modifications may further be made without departing from the principle of the present invention. These improvements and modifications should also be deemed as falling within the protection scope of the present invention.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

1. A kit for detecting a subset of a human regulatory T cell, comprising: a blood dilution solution, a mononuclear cell isolation solution, a cell culture solution, a lymphocyte activation liquid, a dead-cell-removing dye, an FcR blocking agent, fluorescence-labeled antibodies against human cell-surface markers, fluorescence-labeled antibodies against human intracellular molecules, lipopolysaccharide, a washing buffer, a cell staining buffer, a cell fixing solution, and a membrane-permeable wash buffer; wherein the fluorescence-labeled antibodies against human cell-surface markers comprise an anti-human CD3 or CD4 antibody, an anti-human CD8 antibody, an anti-human CD25 antibody, an anti-human CD127 antibody, an anti-human ICOS antibody, an anti-human TGF-β antibody, and an anti-human CCR6 antibody; and wherein the fluorescence-labeled antibodies against human intracellular molecules comprise an anti-human IL-10 antibody, an anti-human IL-17 antibody, and an anti-human Foxp3 antibody.

2. The kit according to claim 1, wherein the fluorescence species of the dead-cell-removing dye, the fluorescence-labeled antibodies against human cell-surface marker molecules, and the fluorescence species of the fluorescence-labeled antibodies against human intracellular molecules comprise Alexa Fluor 488, Alexa Fluor 594, Alexa Fluor 647, Alexa Fluor 700, APC, APC/Cy7, APC/H7, APC/R700, BB515, BB700, BD IMag/DM, BUV395, BUV496, BUV563, BUV661, BUV737, BUV805, Brilliant Violet 421, Brilliant Violet 480, Brilliant Violet 510, Brilliant Blue 515, Brilliant Violet 570, Brilliant Violet 605, Brilliant Violet 650, Brilliant Violet711, Brilliant Violet750, Brilliant Violet785, Brilliant Violet786, FITC, NA/LE, LEAF, Pacific Blue, PE, PE/CF594, PE/Cy5, PE/Cy7, PE/Dazzle, PerCP, PerCP/Cy 5.5, V450 or V500.

3. The kit according to claim 2, wherein the fluorescence species can be freely combined according to the laser and optical filter configurations of a flowcytometer.

4. The kit according to claim 1, wherein the blood dilution solution comprises a solution having an osmotic pressure equal to that of human plasma.

5. The kit according to claim 4, wherein the blood dilution solution comprises a NaCl solution or a PBS buffer.

6. The kit according to claim 1, wherein the active ingredients of the mononuclear cell isolation solution comprise: polysucrose and meglumine diatriazoate, or sodium diatrizoate and polysaccharide, or iodixanol.

7. The kit according to claim 1, wherein the active ingredients of the cell fixing solution comprise formaldehyde and methanol.

8. The kit according to claim 1, wherein the active ingredients of the membrane-permeable wash buffer comprise saponin and fetal bovine serum, or saponin and bovine serum albumin.

9. The kit according to claim 1, wherein the active ingredients of the FcR blocker comprise a human immunoglobulin, or an unrelated immunoglobulin which is of the same genus and same subset as those of the antibody used in the flow cytometry.

10. A method for detecting a subset of a human regulatory T cell using the kit according to claim 1, comprising the steps of:
1) mixing venous blood with the blood dilution solution and then placing the mixture onto the upper layer of the mononuclear cell isolation solution, centrifuging for 15-35 min, and then aspirating mononuclear cells, wherein the volume ratio of the venous blood, the blood dilution solution and the mononuclear cell isolation solution is 1:(0.5-2):(0.5-2);
2) diluting the mononuclear cells obtained in step 1) with the blood dilution solution, and then centrifuging at 90-600 g for 4-30 min to collect the cell pellet;
3) resuspending the cell pellet obtained in step 2) with the cell culture medium, and adjusting the cell density to 0.5×10⁶-4×10⁶ per mL, so as to obtain the first cell suspension;
4) uniformly mixing the first cell suspension obtained in step 3) with the lymphocyte activation solution and the lipopolysaccharide, and placing the mixture into and cultivating in an incubator containing CO₂ for 4-6 h, to obtain a first incubation cell;
5) mixing the first incubation cell obtained in step 4) with the PBS buffer according to a volume ratio of 1:(0.5-10), then centrifuging at 90-600 g for 4-30 min, and discarding the supernatant to obtain a second cell suspension;
6) taking the second cell suspension obtained in step 5) and mixing it with 0.1-10 μL of DMSO solution containing the dead-cell-removing dye and 0.1-10 μL of the FcR blocker according to 1×10⁵-10×10⁶ cells per 100 μL of volume, and then incubating at 4-40° C. for 5-30 min, so as to obtain a second incubation cell;
7) mixing the second incubation cell obtained in step 6) with 0.5-5 mL of the cell staining buffer according to 1×10⁵-10×10⁶ cells per 100 μL of volume, centrifuging at 90-600 g for 4-30 min, discarding the supernatant, and then adding 0.5-20 μL of each of the fluorescence-labeled antibodies against human cell-surface marker molecules into the cell pellet according to 1×10⁵-10×10⁶ cells per 100 μL of volume, uniformly mixing, and then incubating with protection from light at 4-40° C. for 5-30 min;
8) mixing the third incubation cell obtained in step 7) with 0.5-5 mL of the cell staining buffer according to 1×10⁵-10×10⁶ cells per 100 μL of volume, then centrifuging at 90-600 g for 4-30 min, discarding the supernatant, then adding 0.1-5 mL of the cell fixing solution into the pellet according to 1×10⁵-10×10⁶ cells per 100 μL of volume for resuspension, and incubating with protection from light at 4-40° C. for 10-120 min, so as to obtain resuspension of the third incubation cell;
9) mixing the resuspension of the third incubation cell obtained in step 8) with 0.5-5 mL of the membrane-permeable wash buffer according to 1×10⁵-10×10⁶ cells per 100 μL of volume, centrifuging at 90-600 g for 4-30 min, discarding the supernatant to obtain a pellet of the third incubation cell, and then again adding 0.5-5 mL of the membrane-permeable wash buffer according to 1×10⁵-10×10⁶ cells per 100 μL of volume to resuspend the pellet of the third incubation cell, centrifuging at 90-600 g for 4-30 min, and discarding the supernatant to obtain a pellet of the third incubation cell;
10) adding 0.5-20 μL of each of the fluorescence-labeled antibodies against human intracellular molecules into the pellet of the third incubation cell obtained in step 9) according to 1×10⁵-10×10⁶ cells per 100 μL of volume, uniformly mixing, and then incubating with protection from light at 4-40° C. for 10-120 min, so as to obtain a fourth incubation cell; and
11) adding 0.5-5 mL of the membrane-permeable washing buffer into the pellet of the fourth incubation cell obtained in step 10) according to 1×10⁵-10×10⁶ cells per 100 μL of volume, uniformly mixing, centrifuging at 90-600 g for 4-30 min, and discarding the supernatant to obtain a pellet of the fourth incubation cell, adjusting the cell density to (0.1-10)×10⁶ per mL with PBS, and then detecting the antibody expression by a flow cytometer; and obtaining the detection result of the Treg cell subsets according to the antibody expression, wherein the correspondence relationship between the Treg cell subsets and the antibody expression is as follows:

| | | Expression conditions of |
|---|---|---|
| | Subset of Treg Cell | antibodies |
| | Inductive (or adaptive) Treg (iTreg) | CD4⁺ CD25⁺Foxp3⁺ |
| | | IL-10⁺ TGF-β⁺ |
| | CD127 low-expression and negative Treg | CD4⁺ CD25⁺Foxp3⁺ |
| | (CD127^{(low&-)} Treg) | CD127^{(low&-)} |
| | ICOS positive Treg (ICOS⁺ | CD4⁺ CD25⁺Foxp3⁺ |
| | Treg) | ICOS⁺ |
| 4 | IL-10-producing Treg Type 1 (Tr1) | CD4⁺ CD25⁺Foxp3⁻ |
| | | IL-10⁺ |
| | CD8 positive Treg (CD8⁺ Treg) | CD8⁺ CD25⁺ Foxp3⁺ |
| | IL-17-producing Treg (IL-17⁺ | CD4⁺ Foxp3⁺ CCR6⁺ |
| | Treg) | IL-17⁺ |

11. A method for detecting a subset of a human regulatory T cell using the kit according to claim 2, comprising the steps of:
1) mixing venous blood with the blood dilution solution and then placing the mixture onto the upper layer of the mononuclear cell isolation solution, centrifuging for 15-35 min, and then aspirating mononuclear cells, wherein the volume ratio of the venous blood, the blood dilution solution and the mononuclear cell isolation solution is 1:(0.5-2):(0.5-2);
2) diluting the mononuclear cells obtained in step 1) with the blood dilution solution, and then centrifuging at 90-600 g for 4-30 min to collect the cell pellet
3) resuspending the cell pellet obtained in step 2) with the cell culture medium, and adjusting the cell density to 0.5×10⁶-4×10⁶ per mL, so as to obtain the first cell suspension;
4) uniformly mixing the first cell suspension obtained in step 3) with the lymphocyte activation solution and the lipopolysaccharide, and placing the mixture into and cultivating in an incubator containing CO₂ for 4-6 h, to obtain a first incubation cell;
5) mixing the first incubation cell obtained in step 4) with the PBS buffer according to a volume ratio of 1:(0.5-10), then centrifuging at 90-600 g for 4-30 min, and discarding the supernatant to obtain a second cell suspension;
6) taking the second cell suspension obtained in step 5) and mixing it with 0.1-10 μL of DMSO solution containing the dead-cell-removing dye and 0.1-10 μL of the FcR blocker according to 1×10⁵-10×10⁶ cells per 100 μL of volume, and then incubating at 4-40° C. for 5-30 min, so as to obtain a second incubation cell;
7) mixing the second incubation cell obtained in step 6) with 0.5-5 mL of the cell staining buffer according to 1×10⁵-10×10⁶ cells per 100 μL of volume, centrifuging at 90-600 g for 4-30 min, discarding the supernatant, and then adding 0.5-20 μL of each of the fluorescence-labeled antibodies against human cell-surface marker molecules into the cell pellet according to 1×10⁵-10×10⁶ cells per 100 μL of volume, uniformly mixing, and then incubating with protection from light at 4-40° C. for 5-30 min;
8) mixing the third incubation cell obtained in step 7) with 0.5-5 mL of the cell staining buffer according to 1×10⁵-10×10⁶ cells per 100 μL of volume, then centrifuging at 90-600 g for 4-30 min, discarding the supernatant, then adding 0.1-5 mL of the cell fixing solution into the pellet according to 1×10⁵-10×10⁶ cells per 100 μL of volume for resuspension, and incubating with protection from light at 4-40° C. for 10-120 min, so as to obtain resuspension of the third incubation cell;
9) mixing the resuspension of the third incubation cell obtained in step 8) with 0.5-5 mL of the membrane-permeable wash buffer according to 1×10⁵-10×10⁶ cells per 100 μL of volume, centrifuging at 90-600 g for 4-30 min, discarding the supernatant to obtain a pellet of the third incubation cell, and then again adding 0.5-5 mL of the membrane-permeable wash buffer according to 1×10⁵-10×10⁶ cells per 100 μL of volume to resuspend the pellet of the third incubation cell, centrifuging at 90-600 g for 4-30 min, and discarding the supernatant to obtain a pellet of the third incubation cell;
10) adding 0.5-20 μL of each of the fluorescence-labeled antibodies against human intracellular molecules into the pellet of the third incubation cell obtained in step 9) according to 1×10⁵-10×10⁶ cells per 100 μL of volume, uniformly mixing, and then incubating with protection from light at 4-40° C. for 10-120 min, so as to obtain a fourth incubation cell; and
11) adding 0.5-5 mL of the membrane-permeable washing buffer into the pellet of the fourth incubation cell obtained in step 10) according to 1×10⁵-10×10⁶ cells per 100 μL of volume, uniformly mixing, centrifuging at 90-600 g for 4-30 min, and discarding the supernatant to obtain a pellet of the fourth incubation cell, adjusting the cell density to (0.1-10)×10⁶ per mL with PBS, and then detecting the antibody expression by a flow cytometer; and obtaining the detection result of the Treg cell subsets according to the antibody expression, wherein the correspondence relationship between the Treg cell subsets and the antibody expression is as follows:

| | Expression conditions of |
|---|---|
| Subset of Treg Cell | antibodies |
| Inductive (or adaptive) Treg (iTreg) | CD4⁺ CD25⁺Foxp3⁺ |
| | IL-10⁺ TGF-β⁺ |
| CD127 low-expression and negative Treg | CD4⁺ CD25⁺Foxp3⁺ |
| (CD127^{(low&-)} Treg) | CD127^{(low&-)} |
| ICOS positive Treg (ICOS⁺ | CD4⁺ CD25⁺Foxp3⁺ |
| Treg) | ICOS⁺ |
| IL-10-producing Treg Type 1 (Tr1) | CD4⁺ CD25⁺Foxp3⁻ |
| | IL-10⁺ |
| CD8 positive Treg (CD8⁺ | CD8⁺ CD25⁺ Foxp3⁺ |
| Treg) | |
| IL-17-producing Treg (IL-17⁺ T | CD4⁺ Foxp3⁺ CCR6⁺ |
| reg) | IL-17⁺ |

12. A method for detecting a subset of a human regulatory T cell using the kit according to claim 3, comprising the steps of:
1) mixing venous blood with the blood dilution solution and then placing the mixture onto the upper layer of the mononuclear cell isolation solution, centrifuging for 15-35 min, and then aspirating mononuclear cells, wherein the volume ratio of the venous blood, the blood dilution solution and the mononuclear cell isolation solution is 1:(0.5-2):(0.5-2);
2) diluting the mononuclear cells obtained in step 1) with the blood dilution solution, and then centrifuging at 90-600 g for 4-30 min to collect the cell pellet
3) resuspending the cell pellet obtained in step 2) with the cell culture medium, and adjusting the cell density to 0.5×10⁶-4×10⁶ per mL, so as to obtain the first cell suspension;
4) uniformly mixing the first cell suspension obtained in step 3) with the lymphocyte activation solution and the lipopolysaccharide, and placing the mixture into and cultivating in an incubator containing CO₂ for 4-6 h, to obtain a first incubation cell;
5) mixing the first incubation cell obtained in step 4) with the PBS buffer according to a volume ratio of 1:(0.5-10), then centrifuging at 90-600 g for 4-30 min, and discarding the supernatant to obtain a second cell suspension;
6) taking the second cell suspension obtained in step 5) and mixing it with 0.1-10 μL of DMSO solution containing the dead-cell-removing dye and 0.1-10 μL of the FcR blocker according to 1×10⁵-10×10⁶ cells per 100 μL of volume, and then incubating at 4-40° C. for 5-30 min, so as to obtain a second incubation cell;
7) mixing the second incubation cell obtained in step 6) with 0.5-5 mL of the cell staining buffer according to 1×10⁵-10×10⁶ cells per 100 μL of volume, centrifuging at 90-600 g for 4-30 min, discarding the supernatant, and then adding 0.5-20 μL of each of the fluorescence-labeled antibodies against human cell-surface marker molecules into the cell pellet according to 1×10⁵-10×10⁶ cells per 100 μL of volume, uniformly mixing, and then incubating with protection from light at 4-40° C. for 5-30 min;
8) mixing the third incubation cell obtained in step 7) with 0.5-5 mL of the cell staining buffer according to 1×10⁵-10×10⁶ cells per 100 μL of volume, then centrifuging at 90-600 g for 4-30 min, discarding the supernatant, then adding 0.1-5 mL of the cell fixing solution into the pellet according to 1×10⁵-10×10⁶ cells per 100 μL of volume for resuspension, and incubating with protection from light at 4-40° C. for 10-120 min, so as to obtain resuspension of the third incubation cell;
9) mixing the resuspension of the third incubation cell obtained in step 8) with 0.5-5 mL of the membrane-permeable wash buffer according to 1×10⁵-10×10⁶ cells per 100 μL of volume, centrifuging at 90-600 g for 4-30 min, discarding the supernatant to obtain a pellet of the third incubation cell, and then again adding 0.5-5 mL of the membrane-permeable wash buffer according to 1×10⁵-10×10⁶ cells per 100 μL of volume to resuspend the pellet of the third incubation cell, centrifuging at 90-600 g for 4-30 min, and discarding the supernatant to obtain a pellet of the third incubation cell;
10) adding 0.5-20 μL of each of the fluorescence-labeled antibodies against human intracellular molecules into the pellet of the third incubation cell obtained in step 9) according to 1×10⁵-10×10⁶ cells per 100 μL of volume, uniformly mixing, and then incubating with protection from light at 4-40° C. for 10-120 min, so as to obtain a fourth incubation cell; and
11) adding 0.5-5 mL of the membrane-permeable washing buffer into the pellet of the fourth incubation cell obtained in step 10) according to 1×10⁵-10×10⁶ cells per 100 μL of volume, uniformly mixing, centrifuging at 90-600 g for 4-30 min, and discarding the supernatant to obtain a pellet of the fourth incubation cell, adjusting the cell density to (0.1-10)×10⁶ per mL with PBS, and then detecting the antibody expression by a flow cytometer; and obtaining the detection result of the Treg cell subsets according to the antibody expression, wherein the correspondence relationship between the Treg cell subsets and the antibody expression is as follows:

| | Expression conditions of |
|---|---|
| Subset of Treg Cell | antibodies |
| Inductive (or adaptive) Treg (iTreg) | CD4⁺ CD25⁺Foxp3⁺ |
| | IL-10⁺ TGF-β⁺ |
| CD127 low-expression and negative Treg | CD4⁺ CD25⁺Foxp3⁺ |
| (CD127^{(low&-)} Treg) | CD127^{(low&-)} |
| ICOS positive Treg (ICOS⁺ | CD4⁺ CD25⁺Foxp3⁺ |
| Treg) | ICOS⁺ |
| IL-10-producing Treg Type 1 (Tr1) | CD4⁺ CD25⁺Foxp3⁻ |
| | IL-10⁺ |
| CD8 positive Treg (CD8⁺ Treg) | CD8⁺ CD25⁺ Foxp3⁺ |
| IL-17-producing Treg (IL-17⁺ | CD4⁺ Foxp3⁺ CCR6⁺ |
| Treg) | IL-17⁺ |

13. The method for detecting a subset of a human regulatory T cell according to claim 10, wherein the cell culture solution of step 3) is based on an RPMI 1640 medium and comprises fetal bovine serum at a volume concentration of 0.1%-20% and a penicillin-streptomycin double-resistant solution at a volume concentration of 0-3%.

14. The method for detecting a subset of a human regulatory T cell according to claim 10, wherein the incubation conditions of step 4) comprise: incubating in an incubator containing 5% CO₂ at an incubation temperature of 35-40° C.

15. The method for detecting a subset of a human regulatory T cell according to claim 10, wherein the addition volume of each one of the fluorescence-labeled antibodies against human cell-surface markers and fluorescence-labeled antibodies against human intracellular molecules of step 7) and step 9) is determined according to the number of incubation cells and the working concentration of the antibody.

16. A method for detecting a subset of a human regulatory T cell using the kit according to claim 4, comprising the steps of:
1) mixing venous blood with the blood dilution solution and then placing the mixture onto the upper layer of the mononuclear cell isolation solution, centrifuging for 15-35 min, and then aspirating mononuclear cells, wherein the volume ratio of the venous blood, the blood dilution solution and the mononuclear cell isolation solution is 1:(0.5-2):(0.5-2);
2) diluting the mononuclear cells obtained in step 1) with the blood dilution solution, and then centrifuging at 90-600 g for 4-30 min to collect the cell pellet;
3) resuspending the cell pellet obtained in step 2) with the cell culture medium, and adjusting the cell density to 0.5×10⁶-4×10⁶ per mL, so as to obtain the first cell suspension;
4) uniformly mixing the first cell suspension obtained in step 3) with the lymphocyte activation solution and the lipopolysaccharide, and placing the mixture into and cultivating in an incubator containing CO₂ for 4-6 h, to obtain a first incubation cell;
5) mixing the first incubation cell obtained in step 4) with the PBS buffer according to a volume ratio of 1:(0.5-10), then centrifuging at 90-600 g for 4-30 min, and discarding the supernatant to obtain a second cell suspension;
6) taking the second cell suspension obtained in step 5) and mixing it with 0.1-10 μL of DMSO solution containing the dead-cell-removing dye and 0.1-10 μL of the FcR blocker according to 1×10⁵-10×10⁶ cells per 100 μL of volume, and then incubating at 4-40° C. for 5-30 min, so as to obtain a second incubation cell;
7) mixing the second incubation cell obtained in step 6) with 0.5-5 mL of the cell staining buffer according to 1×10⁵-10×10⁶ cells per 100 μL of volume, centrifuging at 90-600 g for 4-30 min, discarding the supernatant, and then adding 0.5-20 μL of each of the fluorescence-labeled antibodies against human cell-surface marker molecules into the cell pellet according to 1×10⁵-10×10⁶ cells per 100 μL of volume, uniformly mixing, and then incubating with protection from light at 4-40° C. for 5-30 min;
8) mixing the third incubation cell obtained in step 7) with 0.5-5 mL of the cell staining buffer according to 1×10⁵-10×10⁶ cells per 100 μL of volume, then centrifuging at 90-600 g for 4-30 min, discarding the supernatant, then adding 0.1-5 mL of the cell fixing solution into the pellet according to 1×10⁵-10×10⁶ cells per 100 μL of volume for resuspension, and incubating with protection from light at 4-40° C. for 10-120 min, so as to obtain resuspension of the third incubation cell;
9) mixing the resuspension of the third incubation cell obtained in step 8) with 0.5-5 mL of the membrane-permeable wash buffer according to 1×10⁵-10×10⁶ cells per 100 μL of volume, centrifuging at 90-600 g for 4-30 min, discarding the supernatant to obtain a pellet of the third incubation cell, and then again adding 0.5-5 mL of the membrane-permeable wash buffer according to 1×10⁵-10×10⁶ cells per 100 μL of volume to resuspend the pellet of the third incubation cell, centrifuging at 90-600 g for 4-30 min, and discarding the supernatant to obtain a pellet of the third incubation cell;
10) adding 0.5-20 μL of each of the fluorescence-labeled antibodies against human intracellular molecules into the pellet of the third incubation cell obtained in step 9) according to 1×10⁵-10×10⁶ cells per 100 μL of volume, uniformly mixing, and then incubating with protection from light at 4-40° C. for 10-120 min, so as to obtain a fourth incubation cell; and
11) adding 0.5-5 mL of the membrane-permeable washing buffer into the pellet of the fourth incubation cell obtained in step 10) according to 1×10⁵-10×10⁶ cells per 100 μL of volume, uniformly mixing, centrifuging at 90-600 g for 4-30 min, and discarding the supernatant to obtain a pellet of the fourth incubation cell, adjusting the cell density to (0.1-10)×10⁶ per mL with PBS, and then detecting the antibody expression by a flow cytometer; and obtaining the detection result of the Treg cell subsets according to the antibody expression, wherein the correspondence relationship between the Treg cell subsets and the antibody expression is as follows:

| | Expression conditions of |
|---|---|
| Subset of Treg Cell | antibodies |
| Inductive (or adaptive) Treg (iTreg) | CD4⁺ CD25⁺Foxp3⁺ |
| | IL-10⁺ TGF-β⁺ |
| CD127 low-expression and negative Treg | CD4⁺ CD25⁺Foxp3⁺ |
| (CD127^{(low&-)} Treg) | CD127^{(low&-)} |
| ICOS positive Treg (ICOS⁺ | CD4⁺ CD25⁺Foxp3⁺ |
| Treg) | ICOS⁺ |
| IL-10-producing Treg Type 1 (Tr1) | CD4⁺ CD25⁺Foxp3⁻ |
| | IL-10⁺ |
| CD8 positive Treg (CD8⁺ | CD8⁺ CD25⁺ Foxp3⁺ |
| Treg) | |
| IL-17-producing Treg (IL-17⁺ | CD4⁺ Foxp3⁺ CCR6⁺ |
| Treg) | IL-17⁺ |

17. A method for detecting a subset of a human regulatory T cell using the kit according to claim 5, comprising the steps of:
1) mixing venous blood with the blood dilution solution and then placing the mixture onto the upper layer of the mononuclear cell isolation solution, centrifuging for 15-35 min, and then aspirating mononuclear cells, wherein the volume ratio of the venous blood, the blood dilution solution and the mononuclear cell isolation solution is 1:(0.5-2):(0.5-2);
2) diluting the mononuclear cells obtained in step 1) with the blood dilution solution, and then centrifuging at 90-600 g for 4-30 min to collect the cell pellet;
3) resuspending the cell pellet obtained in step 2) with the cell culture medium, and adjusting the cell density to 0.5×10⁶-4×10⁶ per mL, so as to obtain the first cell suspension;
4) uniformly mixing the first cell suspension obtained in step 3) with the lymphocyte activation solution and the lipopolysaccharide, and placing the mixture into and cultivating in an incubator containing CO₂ for 4-6 h, to obtain a first incubation cell;
5) mixing the first incubation cell obtained in step 4) with the PBS buffer according to a volume ratio of 1:(0.5-10), then centrifuging at 90-600 g for 4-30 min, and discarding the supernatant to obtain a second cell suspension;
6) taking the second cell suspension obtained in step 5) and mixing it with 0.1-10 μL of DMSO solution containing the dead-cell-removing dye and 0.1-10 μL of the FcR blocker according to 1×10⁵-10×10⁶ cells per 100 μL of volume, and then incubating at 4-40° C. for 5-30 min, so as to obtain a second incubation cell;
7) mixing the second incubation cell obtained in step 6) with 0.5-5 mL of the cell staining buffer according to 1×10⁵-10×10⁶ cells per 100 μL of volume, centrifuging at 90-600 g for 4-30 min, discarding the supernatant, and then adding 0.5-20 μL of each of the fluorescence-labeled antibodies against human cell-surface marker molecules into the cell pellet according to 1×10⁵-10×10⁶ cells per 100 μL of volume, uniformly mixing, and then incubating with protection from light at 4-40° C. for 5-30 min;
8) mixing the third incubation cell obtained in step 7) with 0.5-5 mL of the cell staining buffer according to 1×10⁵-10×10⁶ cells per 100 μL of volume, then centrifuging at 90-600 g for 4-30 min, discarding the supernatant, then adding 0.1-5 mL of the cell fixing solution into the pellet according to 1×10⁵-10×10⁶ cells per 100 μL of volume for resuspension, and incubating with protection from light at 4-40° C. for 10-120 min, so as to obtain resuspension of the third incubation cell;
9) mixing the resuspension of the third incubation cell obtained in step 8) with 0.5-5 mL of the membrane-permeable wash buffer according to 1×10⁵-10×10⁶ cells per 100 μL of volume, centrifuging at 90-600 g for 4-30 min, discarding the supernatant to obtain a pellet of the third incubation cell, and then again adding 0.5-5 mL of the membrane-permeable wash buffer according to 1×10⁵-10×10⁶ cells per 100 μL of volume to resuspend the pellet of the third incubation cell, centrifuging at 90-600 g for 4-30 min, and discarding the supernatant to obtain a pellet of the third incubation cell;
10) adding 0.5-20 μL of each of the fluorescence-labeled antibodies against human intracellular molecules into the pellet of the third incubation cell obtained in step 9) according to 1×10⁵-10×10⁶ cells per 100 μL of volume, uniformly mixing, and then incubating with protection from light at 4-40° C. for 10-120 min, so as to obtain a fourth incubation cell; and
11) adding 0.5-5 mL of the membrane-permeable washing buffer into the pellet of the fourth incubation cell obtained in step 10) according to 1×10⁵-10×10⁶ cells per 100 μL of volume, uniformly mixing, centrifuging at 90-600 g for 4-30 min, and discarding the supernatant to obtain a pellet of the fourth incubation cell, adjusting the cell density to (0.1-10)×10⁶ per mL with PBS, and then detecting the antibody expression by a flow cytometer; and obtaining the detection result of the Treg cell subsets according to the antibody expression, wherein the correspondence relationship between the Treg cell subsets and the antibody expression is as follows:

| | Expression conditions of |
|---|---|
| Subset of Treg Cell | antibodies |
| Inductive (or adaptive) Treg (iTreg) | CD4⁺ CD25⁺Foxp3⁺ |
| | IL-10⁺ TGF-β⁺ |
| CD127 low-expression and negative Treg | CD4⁺ CD25⁺Foxp3⁺ |
| (CD127^{(low&-)} Treg) | CD127^{(low&-)} |
| ICOS positive Treg (ICOS⁺ | CD4⁺ CD25⁺Foxp3⁺ |
| Treg) | ICOS⁺ |
| IL-10-producing Treg Type 1 (Tr1) | CD4⁺ CD25⁺Foxp3⁻ |
| | IL-10⁺ |
| CD8 positive Treg (CD8⁺ | CD8⁺ CD25⁺ Foxp3⁺ |
| Treg) | |
| IL-17-producing Treg (IL-17⁺ | CD4⁺ Foxp3⁺ CCR6⁺ |
| Treg) | IL-17⁺ |

18. A method for detecting a subset of a human regulatory T cell using the kit according to claim 6, comprising the steps of:
1) mixing venous blood with the blood dilution solution and then placing the mixture onto the upper layer of the mononuclear cell isolation solution, centrifuging for 15-35 min, and then aspirating mononuclear cells, wherein the volume ratio of the venous blood, the blood dilution solution and the mononuclear cell isolation solution is 1:(0.5-2):(0.5-2);
2) diluting the mononuclear cells obtained in step 1) with the blood dilution solution, and then centrifuging at 90-600 g for 4-30 min to collect the cell pellet;
3) resuspending the cell pellet obtained in step 2) with the cell culture medium, and adjusting the cell density to 0.5×10⁶-4×10⁶ per mL, so as to obtain the first cell suspension;
4) uniformly mixing the first cell suspension obtained in step 3) with the lymphocyte activation solution and the lipopolysaccharide, and placing the mixture into and cultivating in an incubator containing CO₂ for 4-6 h, to obtain a first incubation cell;
5) mixing the first incubation cell obtained in step 4) with the PBS buffer according to a volume ratio of 1:(0.5-10), then centrifuging at 90-600 g for 4-30 min, and discarding the supernatant to obtain a second cell suspension;
6) taking the second cell suspension obtained in step 5) and mixing it with 0.1-10 μL of DMSO solution containing the dead-cell-removing dye and 0.1-10 μL of the FcR blocker according to 1×10⁵-10×10⁶ cells per 100 μL of volume, and then incubating at 4-40° C. for 5-30 min, so as to obtain a second incubation cell;
7) mixing the second incubation cell obtained in step 6) with 0.5-5 mL of the cell staining buffer according to 1×10⁵-10×10⁶ cells per 100 μL of volume, centrifuging at 90-600 g for 4-30 min, discarding the supernatant, and then adding 0.5-20 μL of each of the fluorescence-labeled antibodies against human cell-surface marker molecules into the cell pellet according to 1×10⁵-10×10⁶ cells per 100 μL of volume, uniformly mixing, and then incubating with protection from light at 4-40° C. for 5-30 min;
8) mixing the third incubation cell obtained in step 7) with 0.5-5 mL of the cell staining buffer according to 1×10⁵-10×10⁶ cells per 100 μL of volume, then centrifuging at 90-600 g for 4-30 min, discarding the supernatant, then adding 0.1-5 mL of the cell fixing solution into the pellet according to 1×10⁵-10×10⁶ cells per 100 μL of volume for resuspension, and incubating with protection from light at 4-40° C. for 10-120 min, so as to obtain resuspension of the third incubation cell;
9) mixing the resuspension of the third incubation cell obtained in step 8) with 0.5-5 mL of the membrane-permeable wash buffer according to 1×10⁵-10×10⁶ cells per 100 μL of volume, centrifuging at 90-600 g for 4-30 min, discarding the supernatant to obtain a pellet of the third incubation cell, and then again adding 0.5-5 mL of the membrane-permeable wash buffer according to 1×10⁵-10×10⁶ cells per 100 μL of volume to resuspend the pellet of the third incubation cell, centrifuging at 90-600 g for 4-30 min, and discarding the supernatant to obtain a pellet of the third incubation cell;
10) adding 0.5-20 μL of each of the fluorescence-labeled antibodies against human intracellular molecules into the pellet of the third incubation cell obtained in step 9) according to 1×10⁵-10×10⁶ cells per 100 μL of volume, uniformly mixing, and then incubating with protection from light at 4-40° C. for 10-120 min, so as to obtain a fourth incubation cell; and
11) adding 0.5-5 mL of the membrane-permeable washing buffer into the pellet of the fourth incubation cell obtained in step 10) according to 1×10⁵-10×10⁶ cells per 100 μL of volume, uniformly mixing, centrifuging at 90-600 g for 4-30 min, and discarding the supernatant to obtain a pellet of the fourth incubation cell, adjusting the cell density to (0.1-10)×10⁶ per mL with PBS, and then detecting the antibody expression by a flow cytometer; and obtaining the detection result of the Treg cell subsets according to the antibody expression, wherein the correspondence relationship between the Treg cell subsets and the antibody expression is as follows:

| | Expression conditions of |
|---|---|
| Subset of Treg Cell | antibodies |
| Inductive (or adaptive) Treg (iTreg) | CD4⁺ CD25⁺Foxp3⁺ |
| | IL-10⁺ TGF-β⁺ |
| CD127 low-expression and negative Treg | CD4⁺ CD25⁺Foxp3⁺ |
| (CD127^{(low&-)} Treg) | CD127^{(low&-)} |
| ICOS positive Treg (ICOS⁺ | CD4⁺ CD25⁺Foxp3⁺ |
| Treg) | ICOS⁺ |
| IL-10-producing Treg Type 1 (Tr1) | CD4⁺ CD25⁺Foxp3⁻ |
| | IL-10⁺ |
| CD8 positive Treg (CD8⁺ | CD8⁺ CD25⁺ Foxp3⁺ |
| Treg) | |
| IL-17-producing Treg (IL-17⁺ T | CD4⁺ Foxp3⁺ CCR6⁺ |
| reg) | IL-17⁺ |

19. The method for detecting a subset of a human regulatory T cell according to claim 10, wherein the centrifugal conditions of step 1) comprises: a centrifugal force of 200-1000 g, and a centrifugal temperature of 18-22° C.

20. The method for detecting a subset of a human regulatory T cell according to claims 10, wherein in step 2) the volume ratio of the mononuclear cell to the blood dilution solution is 1:(0.5-50).
